(19) United States
(12) Patent Application Publication
(10) Pub. No.: US 2014/0220556 A1
(43) Pub. Date: Aug. 7, 2014

(54) Method of Design and Synthesis of a New Drug

(71) Applicants: Artur Martynov, Kharkov (UA); Boris S. Farber, Brooklyn, NY (US); Sonya Sophya Farber, New York, NY (US)

(72) Inventors: Artur Martynov, Kharkov (UA); Boris S. Farber, Brooklyn, NY (US); Sonya Sophya Farber, New York, NY (US)

(21) Appl. No.: 13/761,103

(22) Filed: Feb. 6, 2013

(51) Int. Cl. C12Q 1/02

(52) U.S. Cl. CPC C12Q 1/025
USPC 435/6.1; 435/32

(57) ABSTRACT

A method of design and synthesis of a new drug. This invention may be used in human and veterinary medicine for the design of new drugs that are effective in the treatment of oncological and viral human and animal illnesses and for the design of new medicines. In the method a biopolymer target for the drug action is selected; then the quantity of nitrogen-containing positively charged groups available for modification is calculated. Biopolymer target may be cut into oligomer fragments. Some of calculated nitrogen-containing positively charged groups are substituted with negatively charged groups by combinatorial modification. The obtained supramolecular assemblies are used as drug for the biopolymer target.

### CROSS REFERENCE TO RELATED APPLICATIONS

The present application is a continuation-in-part of the application Ser. No. 12/931,469, filed Feb. 1, 2011, which is a continuation of the International Application No. PCT/RU2010/000694, filed Nov. 22, 2010.

### TECHNICAL FIELD

This invention relates to medicine and pharmaceuticals, specifically, to methods of design and synthesis of new drugs.

### BACKGROUND OF THE INVENTION

The important place on the modern pharmaceutical market belongs to biotechnological drugs of various origins. These drugs are essential to patients' well-being and include recombinant insulins, interferons, interleukines, erythropoietins, and such. However, the statistical data related to introduction of these and some low-molecular drugs to the market, demonstrate that they are insignificantly more effective than a placebo. For example, the use of beta-interferon (in the treatment of disseminated sclerosis) exceeds the placebo in effectiveness by only 8% (placebo: 30%; beta-interferon: 38%). The situation is almost the same for low-molecular drugs, such as anti-hypertensives. The effectiveness of amlodipin at the third stage of clinical tests was only 22% higher than that of the placebo (amlodipin: 52%; placebo: 30%). The reasons, why a drug might turn out to be ineffective or of little effect for 48% of patients have not yet been determined. The most difficult is to explain the ineffectiveness of drugs, which acceptors are cell receptors that have long been the subjects of study: namely, adreno-, cholino-, and histamine receptors. It is still not understood, why the same drug can be completely ineffective for one group of patience, while remaining effective for another group. Due to this little-studied peculiarity of the human organism, the majority of antihypertensive drugs are taken in combination. It is critical to combine at least three drugs with different mechanisms of action but with the same final result: for example, antihypertensive or cytostatic. In the latter case, the differences between various kinds of tumors are especially marked, not only in sensitivity, but also in the individual peculiarities of a specific tumor and host organism. Even poly-chemotherapy often turns out to be ineffective in the treatment of patients with cancer. The FDA statistics on the third stage of clinical tests of drugs demonstrate low effectiveness of practically all medicinal drugs available on the pharmaceutical market. The average effectiveness of the strongest drug (morphine) is 75%. In other cases, we observe either intolerance and toxic effects or an opposite reaction. Even in narcotic drug applications, only 60% of the people who took them are observed to experience the classical effects. The other group of people, who took, for example, cocaine, suffered a severe headache and dizziness without any signs of anesthesia. This kind of divergence of effects may be caused by polymorphism of the receptor system within human population. Earlier, the structure of receptors was considered to be absolutely conservative and invariable for a single, or sometimes several, animal species. At present, more and more scientists tend to believe that receptors differ in much the same way as do human faces, even within a single species. These differences are caused not only and not so much by the change of the primary amino acid sequence of receptors' protein base as by conformation changes of secondary and tertiary structures. Although they are formally similar in primary structure and molecular weight, different people's receptors are actually different combinations of protein isoforms. This is especially apparent in the example of major histocompatibility complex (MHC) antigen isoform combination. The selection of this complex is vitally important to organ transplantation processes. If thousands of variants and combinations exist for the MHC system, why should the structures of an organism's other receptors be conservative within a species? Most likely, similarly to MHC antigens, the tertiary structures of the majority of cell receptors differ significantly by isoform profile within a population.

This hypothesis provides a good explanation for drugs' low levels of effectiveness. A conservative structure of a classical drug (like one “key”) cannot match a specific receptor (“many different, though similar, locks”) in all individuals of one species equally and with equal affinity.

In order to increase drug effectiveness, it is necessary to change the concept of drug development and approaches to this process. For example, docking [1], one of the most effective methods of modern drug design [2,3] uses a conservative sequence of one receptor. Sometimes target conformations are used in different solvents with further overlay of conformations. It will never be possible to obtain infinitely many receptors and find ONE inhibitor substance for all conformations. One of nature's successful solutions is immunoglobulin system evolution, which protects higher organisms from external aggressive factors, such as viruses, microorganisms, and fungi. Practically the same immunoglobulin base (Fc areas and heavy chains) with a large number of different variants on FAB—fragments being specific to their targets—is quite a successful solution.

In this situation, affinity to targets may vary from 5% of IgM to 95% of IgG. Sometimes one target antigen may cause the generation of several hundred thousand variants of immunoglobulin with different monoclonal specificity for different epitopes. This kind of polymorphism justifies itself: the majority of the population survives infectious diseases. In many cases, convalescent donor immunoglobulins are still the only effective means of treating many diseases, such as SARS and Marburg fever.

Following the logic of nature, we realize that choosing the classical method—synthesis of ONE compound to treat one disease—is irrational and ineffective. This is proven by the decreasing amount of drugs with original structures introduced in the world every year.

To provide the maximum affinity for the maximum number of people, it is necessary to have in one vial a mixture of millions of molecules that are very similar but still differ from each other. In this case we obtain not one “key,” but an entire set of keys.

At least one “key” from this set will match a specific patient and his/her original receptor. If it is unrealistic in modern conditions to synthesize a specific inhibitor for a specific patient, then the only option is to produce millions of inhibitor isoforms in one mole of substance.

In our opinion, one of the most reasonable methods of solving the problem of low drug effectiveness is to obtain precisely partially chemically modified recombinant biotechnological preparations: biopolymers (proteins, polysaccharides, polynucleotides, tannins, self-organized structures/phospholipids, etc.). The usage of this technology will bring pharmaceutical science to a level of intensive development, make molecular modeling methods much simpler and increase the probability of a practical way out for the introduction of drugs.

Initial studies with partial modification of the protein structure were provided with immunoglobulin; the results are presented below. As a rule, except for the primary structure of the compounds, the most important role is played by stereo-chemical properties. Here, the laws of randomness come into play. For example, chloromycetin has two stereoisomeres. Only one of these, the one that rotates to the left, is active.

The picture looks similar with the compounds newly corrected with the help of docking: a great affinity to the protein of the target in the model and the complete lack of activity in the synthesized connections. This is caused by the fact that ideal modeling conditions do not take into account the full variety of influences on the drug—target interaction process. These include parameters such as the temperature; nature, and properties of the solvent; presence of assistive substances—the agents; gravitation; pressure; and, of course, the conformation of the substances. In the majority of cases, it is the stereo-chemical structure of the designed substance that does not coincide with the structure of the synthesized compound. Often, it is impossible to synthesize the necessary substance in general. In order to exclude incorrect conformations during modeling, compounds with a structure that has long been confirmed by physicochemical methods must be used in the capacity of reactants. A contradiction arises again: can this kind of long-known substance inhibit a new target? For these goals the special building elements can be used: proteins and polynucleotides.

The former consist of long-studied L-amino acids, while the later consist of mononucleotides. Monomer structures are well-studied and confirmed, as are their structures within a polymer and the influence of neighboring monomers on each other (this fact is taken into account and used in the process of design and synthesis of primers for a polymerase chain reaction).

Correspondingly, if we look for a drug among peptides and polynucleotides in advance and simply sort out a monomer sequence, we can find the necessary inhibitor or activator with a high degree of probability and also obtain its synthetic derivative.

In this situation, a number of problems arise: how to vary the hydrophobicity and charges of the oligomers obtained and how to protect them from the action of lytic factors in the organism: peptidases and nucleases. Additionally, these compounds must not be large in size; otherwise, they will not be able to get into cells and tissues. One of the methods for modifying protein structure in order to crystallize them better and study the X-ray structures is the acylation of terminal amino groups, lysines, and histidines, by means of various agents, among which the simplest are anhydrides of carboxylic (and polycarboxylic) acids: acetic, succinic, and maleic acids.

The majority of the protein crystals studied using the X-ray structural analysis method are completely succinylated derivatives.

Succinylation leads to a change in molecular charge through the substitution of negatively charged carboxylic groups for positively charged amino groups. If acylation is applied instead of succinylation, not only the molecular charge but also the molecular hydrophobicity level will change.

TABLE 1
Structures of Modified Monomer Amino Acids Formed during Protein
Acylation with Succinic Anhydride

| No. | Monomer Structure | Monomer |
|---|---|---|
| 1 | 2 | Name 3 |
| 1 | 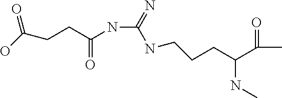 [SMILES: CNC(CCCNC(=N)NC(=O)CCC(=O)O)C(C)=O] | Agrinin succinamide |
| 2 | 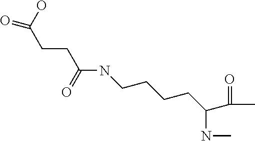 [SMILES: CNC(CCCCNC(=O)CCC(=O)O)C(C)=O] | Lysine succinamide |
| 3 | 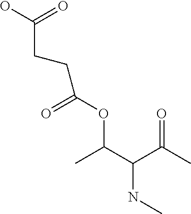 [SMILES: CNC(C(C)=O)C(C)OC(=O)CCC(=O)O] | Ether of threonine and succinic acid |
| 4 | 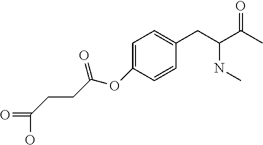 [SMILES: CNC(Cc1ccc(OC(=O)CCC(=O)O)cc1)C(C)=O] | Ether of tyrosine and succinic acid |

Conformation changes of all amino acids in the acylation process by means of succinic and acetic anhydrides are quite well-studied; this eliminates a large portion of the random accidents that may occur during drug design. In this case, the approaches to modeling agonists (activators) and antagonists are completely different from the point of view of receptor polymorphism; this also concerns vaccine design.

There is a classical method of search and obtaining drugs, known as classical empirical screening. This method is characterized by a certain succession of procedures: first, a few thousand chemical compounds are synthesized; the structure of the synthesized compounds is confirmed; a preliminary elimination of inactive compounds is conducted on in vitro screening models. The most active compounds are then studied in animal models. The deficiency of this method is the low percentage of production of active compounds: out of several tens of thousands of synthesized substances, only one, or—two proceed to the clinical research stage. Consequently, not even one of the substances is likely to reach the production stage. In addition to the high volume of resources required for this approach, it is impossible to predict the side effects of synthesized compounds; substances that are highly active at first glance often turn out to be highly toxic. Dozens of years can be spent searching for a single active compound. All compounds synthesized according to this principle are xenobiotics; that is, they have never been encountered in nature before. Accordingly, these substances will cause inestimable damage to the environment when they are excreted from the human body after treatment, since nature does not contain mechanisms to render these substances harmless. In addition, these substances are part of a group of static solid structures, which causes organisms to quickly adapt to the drug: adaptation of microorganisms to antibiotics, of viruses to antiviral drugs and vaccinations, of tumors to antitumor drugs, and so on [4]. For this same reason, the average effectiveness of xenobiotics is slightly higher than that of the placebo and rarely exceeds 50%.

There is another known approach to the design and synthesis of drugs; it has been conditionally named drag design. The method in [5] is the prototype for the invention. It includes a series of procedures: first, the target for drug action is determined; as a rule, this is either a protein or glycoprotein receptor. The given protein is separated, and its structure is established through the use of X-ray structural analysis and NMR spectroscopy. The active center of the protein or the interaction point with potential drugs is determined [5]. With the assistance of molecular computer modeling, the interaction between the target and several drug models is studied [6], drug models are chosen with the most correspondence (affinity) by molecule structure and charge with the active center of the protein (receptor) and the target [7]. Similar procedures also lead to other modifications of the drag design method [8, 9]. Since this mixture of oligomer biopolymers is capable of adaptation and self-organization but is not able to reproduce like a live organism, it has been named a quasi-living system.

Then synthesis and other procedures are conducted as they are in the classic screening method. The advantage in comparison to screening is a significantly less resource-intensive drag design process: just a few dozen substances are synthesized with a significantly higher likelihood of obtaining a truly effective drug (sometimes, up to 20% of the designed and synthesized connections possess the activity claimed). Many of the limitations of classical screening are also applicable to drag design: the high toxicity of the majority of products and the danger of their contamination of the environment, the high adaptability of an organism to a drug, and the ineffectiveness of the drug for up to 50% of the population in connection with the conservative nature of the structure. However, a new problem that has arisen during the use of drag design methods is the non-correspondence of the properties of the modeled and synthesized substances: highly active substances with a maximum level of affinity turn out to be inactive and toxic in practice.

### SUMMARY OF THE INVENTION

This invention was based on the task of developing a new method for the design and synthesis of new, highly effective (with effectiveness in the population of near 100%) drugs that are quickly metabolized in the body and decomposed in nature based on dynamic systems (self-organizing and self-adapting to targets) with minimum development expenses.

The task at hand is addressed through a method of design and synthesis of therapeutic and preventive drugs, including the determination of the drug's target and, using molecular computer modeling methods, and the synthesis of the most active drug possible, distinguished by the fact that in the capacity of a ligand, the same biopolymer target is used, but it is cut into oligomer fragments; or the same, unchanged biopolymer target is used, modified by changing its molecular charge to the opposite charge, with the creation of supramolecular assemblies that are used as active drug. The following are things that can be used in the capacity of a biopolymer target: proteins, or protein mixtures, milk, egg white, DNA, RNA, or a mixture of DNA, RNA, proteins, and combinations thereof. The biopolymer target is cut in fragments with help of proteaseses, nucleases, or synthetic nucleases. The obtained biopolymer target fragments are modified through changing their charge to the opposite charge through acylation with anhydrides of dicarbonate acids or alkylation with halogen-carbon acids. Also, the same biopolymer target may be used as a ligand, but in its native, unchanged state, which is then modified through partially changing the charges of the molecules to the opposite sign with the creation of supramolecular biopolymer assemblies.

We used a supramolecular assembly made of oligomers that were the products of biopolymer hydrolysis, but with molecules charge changed to the opposite, as well as with whole biopolymers with charge partially changed to the opposite. “Assembly” is a term from supramolecular chemistry. The objects of supramolecular chemistry are supramolecular assemblies that build themselves out of their components; i.e. the current geometrical and chemical correspondence of fragments similar to the self-activated collection of the most complex spatial structures in a live cell [10].

Design of Drugs from a Whole Biomolecule—Target with Identified Molecular Structure Resulted into Combinatorial Supramolecular Assemblies:
determination of a biopolymer target for the drug's action;
determination of a quantity of nitrogen-containing groups that are positively charged and available for modification in the biopolymer target;
determination of a a modification rate for the determined quantity of the nitrogen-containing groups;
fragmentation of the biopolymer target into oligomer fragments;
combinatorial modification of the oligomer fragments by substituting a selected number of positively charged nitrogen-containing groups for negatively charged groups, the number is selected according to the modification rate;
application of the modified oligomer fragments as a self-assembled drug that is complementary for the biopolymer target.

With regard to the term “combinatorial supramolecular assemblies” or self-assembled drug it is important to understand the following: Enzymatic cutting of target into small fragments indeed produces one “supramolecular assembly”. However after the partial combinatorial acylation (or alkylation) thousands of fragments appear with different positions of modified groups, which in turn form tens and even hundreds of “supramolecular assemblies”. Combinatorial bioorganic synthesis gives not one, but thousands of different derivatives, which interact not only with target but also with each other.

As it is shown above this method includes new inventive steps that are arranged in a new inventive combination.

Below is an example (FIG. 1) of the combinatorial design of modification rate for the target with two amino groups available for substitution: when to one target molecule (I) two molecules of succinic anhydride (II) are added, their fusion generates only one target molecule with substituted two amide groups (III). However, when to three molecules of the target four modifier molecules are added, fusion results in three different molecules of the target: two molecules with one substituted group in different positions and one fully substituted molecule (IV).

Thus, it is possible to simulate the rate of combinatorial modification of target molecule in order to synthesize thousands of different derivatives (of the same-acylated target, but with different position of substituents and their combinations). The more different derivatives are provided in the reactions, the closer the system will be to living systems and will behave like a self-organizing, self-assembled system. Monomeric components of this assembly of differently modified derivatives of the target can react with each other and engage the non-acylated original target (V) into the reaction of supramolecular self-organization. Thus, the combinatorial part of a modified target, as an assembly, presents a ligand (inhibitor/activator) of the original non-modified target and compiles a drug.

Out of existing calculation methods applied in drug-design, the following methods are applied in our invention: methods of calculating the amino groups available for acylation (amino groups inside of the molecules cannot be modified), and methods of calculating the number of target molecules and the number of acylating agent molecules. Then starts synthesis of oligomers derivatives with partial groups substitution and with formation of supramolecular combinatorial mixtures of molecules. As can be seen in the above FIG. 1, by varying the number of reaction components very different products can be obtained—from inactive fully substituted product (III) to supramolecular assembly (V). As the methods of molecular imaging and calculation of available groups are known, we are claiming the sequence of steps in a new method of drug-design, namely the selection of a target, the selection of an adequate modification rate, the modification of the structure with substitution of amino groups for the carboxyl groups and, accordingly, with change of charges (amino groups to carboxyl groups or to the opposite charges), and the application of the resulting supramolecular assembly as the effector target (ligand: an inhibitor or activator).

An example of such modeling can be the development of a specific inhibitor of valine tRNA, that has powerful anti-cancer properties. Inhibition of tRNA in a cell leads to stop in protein synthesis and thus to cell perish. Thus, the first design step is to choose the target, namely, valine tRNA. The structure of this RNA is well researched and presented in many databases. We know that this RNA has in its structure 18 residues of adenine.

These adenines are attacked by acylating agents in the cold, in water medium. In order to obtain via modification, thousands of fragments with properties of self-organizing structure from a single tRNA molecule, it is necessary to calculate the amount of the modifier and the number of adenines, which must be modified in accordance with the laws of combinatory and combinatorial chemistry. According to the formulas 1 and 2:

m=(2ⁿ−1), (1)

where:

m—number of molecules (and moles) of tRNA, which must be modified to obtain the maximum amount of various tRNA derivatives,

n—number of adenine residues available for modification by anhydride in one tRNA molecule (it is conditionally accepted that tRNA is not hydrolyzed, and represents the whole molecule)

k=n2⁽ⁿ⁻¹⁾ (2)

where:

k—number of moles of succinic anhydride that is necessary for the modification of a tRNA molecule containing n groups available for modification

As a result of calculations we have m=262,143 and k=2359296, which gives the molar ratio of tRNA molecules to anhydride as 1:9. As a result of combinatorial modification of the whole tRNA molecule in this mole ratio there are produced 262,143 different molecules of acylated tRNA derivatives. These molecules are able to react with each other and form complex supramolecular assemblies, find the targets, and include them in their structure with complete inactivation of these targets.

In fact, a system with such variety of elements in the structure behaves as a quasi-living self-organizing system that can actively respond to external factors and reorganize its supramolecular structure.

If prior to its modification tRNA is fragmented by ribonuclease, then, first, four oligomer fragment are formed, and the number of different molecules after adenine acylation increases. Small size of tRNA oligomer fragments allows them to easily penetrate the tissues and cells and inactivate tRNA targets there. The fragmentation of biopolymer to oligomer fragments is needed to obtain good bioavailability of the drug.

If more than one tRNA, but all RNA from cell are used, for example, in yeast cell, the number of different elements in the system would be close to a billion, and such a mixture of oligomer fragments would act as a living system, performing its function—i.e., finding complementary parts in the original RNA and conjugate with them (Antican Preparation). The fact of such a specific conjugation between DNA plasmids and acylated derivatives has been shown earlier.

Design of Drugs from Biopolymers Mixture with Unclear Structure with Induced Combinatorial Supramolecular Assemblies
determination of a quantity of nitrogen-containing groups that are positively charged and available for modification in the biopolymer target (mixture of biopolymers, like milk, or eggs white) by ion changing chromatography for proteins, or identification of adenine and guanine quantity after hydrolysis with the HPLC (n) method;
determination of a modification rate for the determined quantity of the nitrogen-containing groups (k);
fragmentation of the biopolymer target into oligomer fragments;
combinatorial modification of the oligomer fragments by substituting a selected number of positively charged nitrogen-containing groups (k) for negatively charged groups, the number is selected according to the modification rate;
application of the modified oligomer fragments as a self-assembled drug that is complementary for the biopolymer target

In the simulation of biopolymer products with fuzzy, unknown structure, or of biopolymers mixtures with hundred molecules, the calculation of the number of available groups is much more complicated. When mixture of proteins is used their solubility and buffer properties are specifically identified by positively charged residues—lysine and histidines. The quantity of these residues can be calculated by passing a mixture of proteins through an ion exchange anion column.

At the exit of the column the hydrochloric acid is formed, the amount of which is proportional to the number of positively charged nitrogen-containing groups on the protein surface. If the known amount of protein was introduced into the column, the number of groups (n) can be calculated by classical methods. If the target was a mixture of polynucleotides, they should be hydrolyzed first, and the number of adenines and guanines for 1 g of powder should be calculated by using HPLC.

These molecules are modified by alkylation or acylation. Accordingly, the number of groups, based on the total weight, would provide the number of groups (n) available to be modified. Other calculations are provided according to the same formulas that are used for individual biopolymers. Possible variants of modifications and obtaining drugs with fuzzy structure from mixtures of biopolymers are shown in the examples presented below.

### BRIEF DESCRIPTION OF THE DRAWINGS

FIG. 1: The principle of inhibiting the initial target by the acylated target (m=3, k=4)

FIG. 2: Structure of α-2b-Interferon according to Data from the X-Ray-Structural Analysis (online public database of the US National Library of Medicine, Structure 1RH2). Lysine, histidine, and arginine residues are depicted.

FIG. 3: Index of Antiproliferative Activity of IFN Derivatives toward CaOv Cells

FIG. 4: Index of antiproliferative activity of IFN derivatives toward SW-480 Cells

FIG. 5a: Specific Hybridization Of Acylated DNA (RNA) Only With Their Precursors

FIG. 5b: Specific Hybridization Of Acylated DNA plasmids Only With Their Precursors

FIG. 6: Replacement of a Hydrogen Bond with an Ionic Bond in the Creation of Hybrids between Antican and the Target

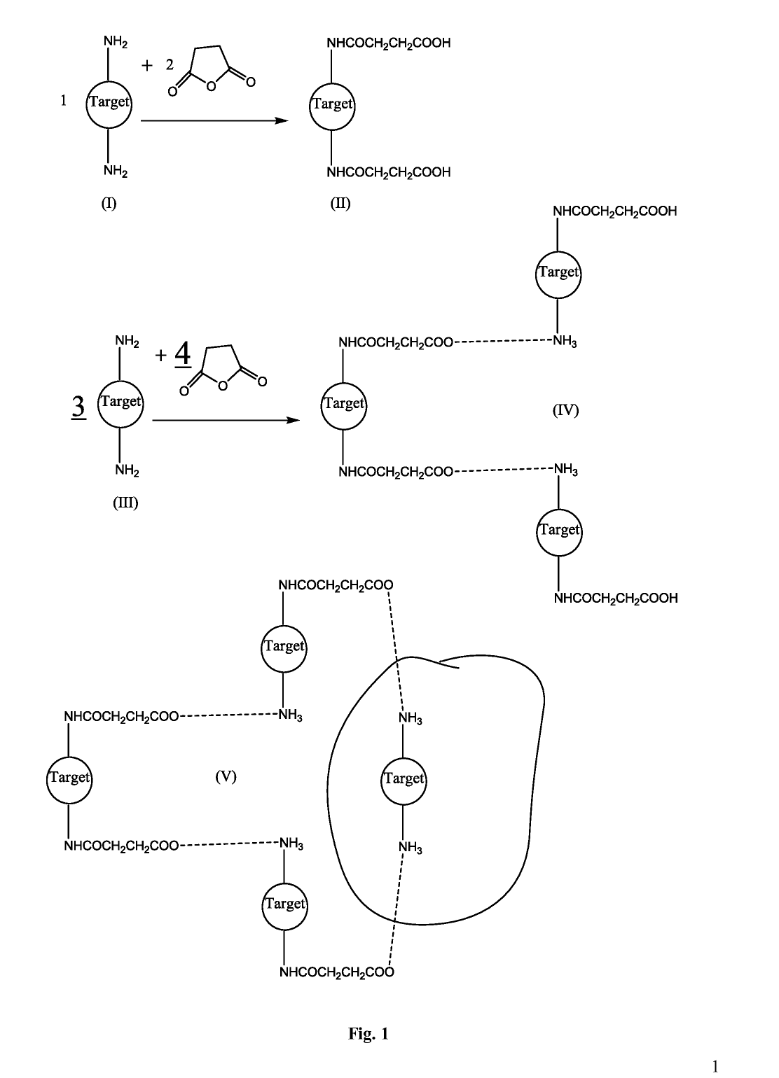

### DESCRIPTION OF THE PREFERRED EXAMPLES

### Example 1

### Synthesis of a Quasi-Living Structure Based on Partially Modified Alpha-Interferon

The average effectiveness of alpha-interferon in the treatment of, for example, cervical cancer, is fairly low (about 15%). The reason for this is the inability of the interferon to interact with the cell receptors. In certain cases, this receptor is blocked by external factors; in other cases, this receptor differs from the ideal in connection with the cancerous transformation of the cells and their mutational changes. To discover the effects of the interferon, we must exchange one individual compound—alpha interferon (the key)—with a mixture of very similar, but distinct molecules of modified interferons (a set of skeleton keys). A selection of “skeleton keys” from hundreds of molecules of interferon can be done through a partial combinatory modification of the structure of alpha interferon.

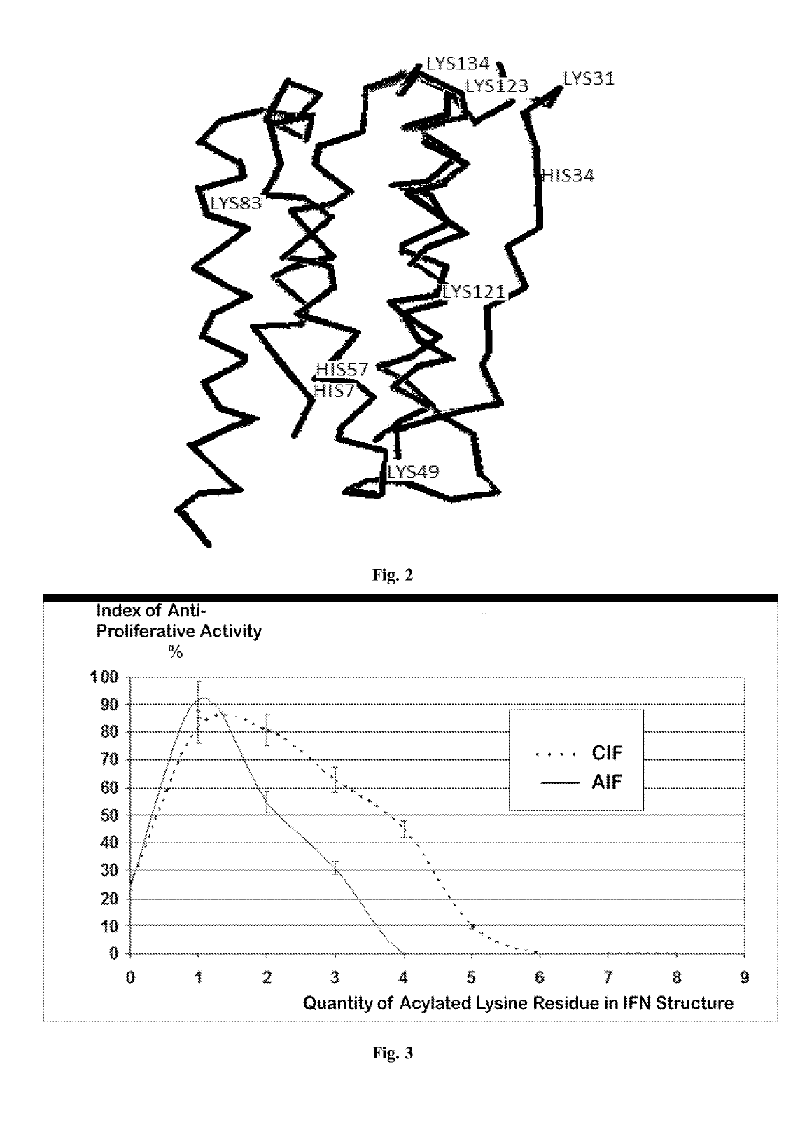

For example, native alpha interferon contains eight fragments of lysine and three fragments of histidine, which can be acylated using succinic anhydride (FIG. 2). In order to obtain a “set of skeleton keys” a mixture of the maximum number of different interferon derivatives must be obtained in one volume with an insignificant change to the active center (area of connection with the receptor): from fully acylated to mono-substituted and mixed with each other in various molecular combinations. This requires determination of the rate of substitution that would facilitate the maximum drug activity in the presence of the maximum quantity of various molecules in one volume of substance.

For alpha-interferon, this is a three-substituted polymorphic derivative. We specifically used the term “polymorphic” in connection with the fact that the solution actually contains 3!*8! interferon derivatives; it contains fully substituted and mono-substituted interferon and a mixture of derivatives with a rate of mixed substitutions greater and less than three. Therefore, partial precision modification allows us to obtain a “set of skeleton keys” to a single polymorphic receptor, which significantly increases the pharmacological activity of the drug in a population of humans.

At minimum, one of the 3!*8! derivatives will correspond with a given receptor and the drug will be biologically active in the given specific instance. In addition, the appearance of these drugs can keep microorganisms and viruses from adapting to the drug. For example, this may take place in the case of bacteriocin modification. This modification may significantly expand the activity spectrum of bacteriocins and increase their protein breakdown stability; this will allow new peptide antibiotics to be created on their basis on an industrial scale.

For the synthesis of quasi-living systems on the basis of α-2b-interferon, interferon (IFN) (Biolek, Kharkiv, Ukraine) and succinic and aconite anhydrides (Aldrich-Sigma, USA) were used. The acylated IFN was made in a weak alkaline buffer solution through adding powdered anhydride in the amounts presented in Tables 1 and 2. The end of the reaction was controlled through changing the molecular weight of the IFN through pulse-gel-electrophoresis in polyacrylamide gel, using non-acylated IFN as a control. The gels were revealed through silver staining [11]. The modified IFN were cleaned with chromatography on Sephadex G-25 and then preserved at −20° C. Their antiproliferative activity was studied on cells of the CaOv line, which is highly sensitive to interferons in vitro, and on SW-480, a cell line with a low sensitivity to the activity of IFN in vitro. The cells were cultivated in a DMEM medium (Aldrich-Sigma, USA) with the addition of 10% FCS and 50 μg/mL gentamycin (Aldrich-Sigma, USA) with a density of 10⁴ cells/cm² in 96-well plates (Bayer) at a temperature of 37° C. in an atmosphere of 5% CO₂. The cell cultures' sensitivity to IFN was evaluated by the level of slowing the cell growth after treatment with the quasi-living IFN derivatives.

The Index of Anti-proliferative Activity (IAA) of IFN (%) was calculated as a ratio between the number of cells in the experimental well and the number of cells in the control multiplied by 100. The non-specific cytotoxic effect was determined through dyeing the cells with trypan blue incubation after a 24-hour incubation of the cells with IFN derivatives. Not one of the interferon derivatives demonstrated non-specific cytotoxicity. The original titer of non-modified IFN was 2.5×10³ ME/mL; IFN and its quasi-living derivatives were added to the medium at a dose of 1000 U/mL (0.05 μL/well); the acylated interferons were added in accordance with the calculation of the concentration of the protein. The cells were incubated for 72 hours with interferon. The number of cells was determined using an inverted microscope (Leica, Germany) every twelve hours.

Interferon is a low-molecular protein with a molecular weight of 8 kDa. It contains eight fragments of lysine and three fragments of histidine [12], which are capable of being acylated by succinic (SA) and aconite (AA) anhydrides. In FIG. 2, the structure of α-2b-interferon is shown.

As may be seen in FIG. 2, free lysines are found on the surface of the molecule and fulfill a buffering function. The receptor zone (the lower part of the IFN molecule in FIG. 2) contains only one lysine and histidine. The amino groups of these amino acids were inaccessible for acylation by anhydrides. The remainders of histidine from the regulatory area of the IFN molecule (the upper part of the molecule in FIG. 2), conversely, take part in the creation of interferon's internal structure. Thus, the histidine amino groups will not take part in the acylation reaction in connection with the steric hindrances. However, the hydroxyl groups of amino acids like threonine and serine can be acylated, as can sulfhydryl amino acids. To prevent the passing of the reaction to the hydroxylic and sulfhydrylic groups, the acylation reaction was conducted in a weakly alkaline medium. At a pH of more than 7.5, deprotonation of free lysine amino groups is observed, and the lysines are significantly stronger nucleophiles than are the hydroxyls and the S-containing groups. Accordingly, when an exact calculation is made of the relationship between IFN and the anhydrides, seven derivatives each may be obtained in whose molecules from one to seven lysine amino groups will be acylated. Tables 1 and 2 present the relationship between the reagents and the results of the analysis of synthesized succinimide and aconite derivatives of α-2b-interferon.

TABLE 1
Results of the Analysis of Succinimide
Derivatives of α-2b-Interferon (SIF)

| Mole Ratio | | Mr, Da | |
|---|---|---|---|
| IFN to Anhydride | Rf | Calculated | Determined |
| 1:1 | 0.490 ± 0.005 | 18359 ± 10 | 18360 ± 50 |
| 1:2 | 0.462 ± 0.005 | 18458 ± 10 | 18460 ± 50 |
| 1:3 | 0.460 ± 0.005 | 18557 ± 10 | 18560 ± 50 |
| 1:4 | 0.456 ± 0.005 | 18656 ± 10 | 18655 ± 50 |
| 1:5 | 0.442 ± 0.005 | 18755 ± 10 | 18750 ± 50 |
| 1:6 | 0.440 ± 0.005 | 18854 ± 10 | 18855 ± 50 |
| 1:7 | 0.436 ± 0.005 | 18953 ± 10 | 18920 ± 50 |
| 0 (Control interferon) | 0.492 ± 0.005 | 18260 ± 10 | 18250 ± 50 |

TABLE 2
Results of the Analysis of Aconite
Derivatives of α-2b-Interferon (AIF)

| Mole Ratio | | Mr, Da | |
|---|---|---|---|
| IFN to Anhydride | Rf | Calculated | Determined |
| 1:1 | 0.489 ± 0.005 | 18280 ± 10 | 18300 ± 50 |
| 1:2 | 0.463 ± 0.005 | 18457 ± 10 | 18470 ± 50 |
| 1:3 | 0.461 ± 0.005 | 18555 ± 10 | 18580 ± 50 |
| 1:4 | 0.455 ± 0.005 | 18634 ± 10 | 18640 ± 50 |
| 1:5 | 0.443 ± 0.005 | 18754 ± 10 | 18760 ± 50 |
| 1:6 | 0.441 ± 0.005 | 18857 ± 10 | 18860 ± 50 |
| 1:7 | 0.438 ± 0.005 | 18956 ± 10 | 18980 ± 50 |
| 0 (Control interferon) | 0.492 ± 0.005 | 18260 ± 10 | 18250 ± 50 |

As may be seen in the table, the calculated and established masses do not differ from each other, which lead to the conclusion of the acylation reaction both in the synthesis of succinic and in the group of aconite IFN derivatives.

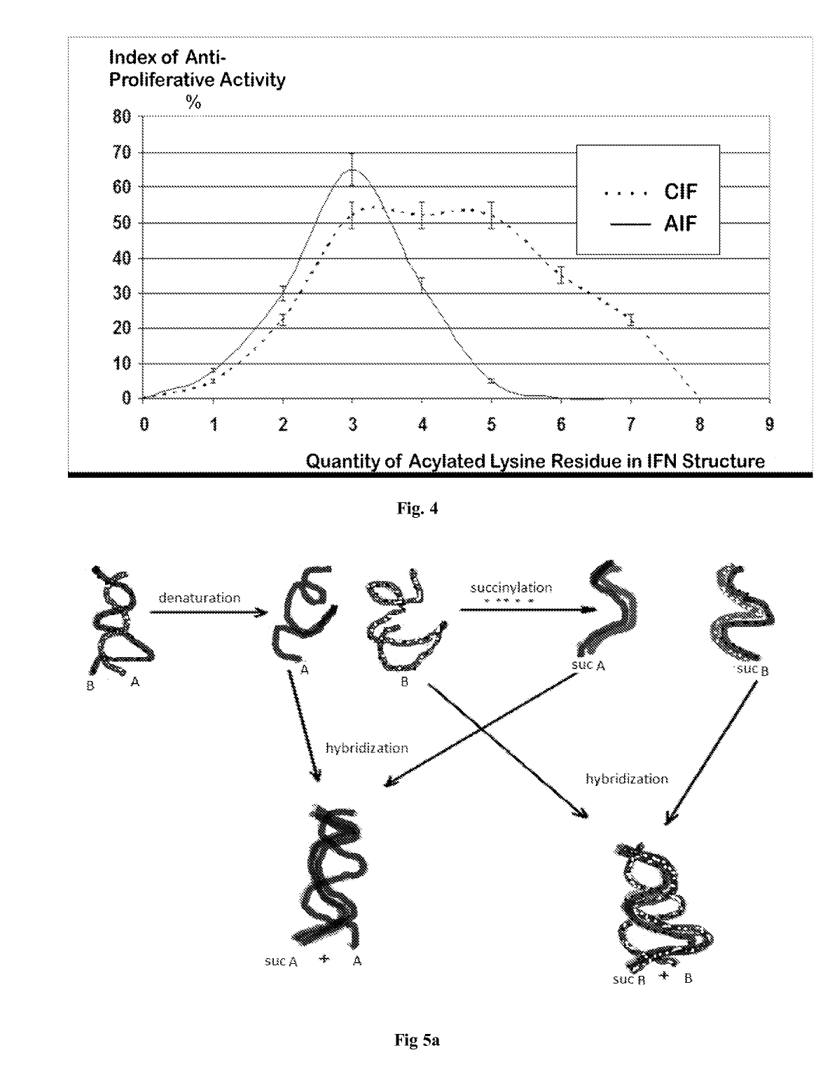

The change in the anti-proliferative effect of the IFN may occur dependent either on its level of affinity with the cell membrane or on the change of the structure of the molecule itself and the corresponding arising of new, previously unknown effects (as phospholipidation of proteins sometimes increases their biological activity dozens of times). Two cultures of human tumor cells were taken for study: one with a high level of sensitivity to interferon and one with a low level of interferon sensitivity. In FIGS. 3 and 4, the results of the study of the anti-proliferative activity of the aconite and succinic IFN derivatives may be seen.

As may be seen in FIG. 3, the acylation of one lysine in the IFN structure by succinic anhydride leads to an increase in the index of anti-proliferation activity (IAA) by 3.2 times. In the derivatives with 2-5 substituted lysines, IAA continues to decrease, and the anti-proliferative activity does not appear in 6-substituted SIFs at all. A slightly different picture is seen with the aconite IFN derivatives: the acylation of one lysine in the IFN by aconite anhydride leads to an increase in anti-proliferation effect of 3.6 times, while similar activity is not seen in the 4-substituted derivatives.

For the SW-480 cultures with low sensitivity to interferon, a different picture can be seen (FIG. 4): the unmodified interferon does not have anti-proliferative activity, while the maximum activity is seen in the derivatives with 3 substituted lysines. The 3-substituted AIF had an activity level 16% higher than that of the SIF. A plateau of stability from three to five substituted lysines was seen in the SIF, while that of the AIF titer began to fall with the substitution of the next lysine.

Thus, a certain pattern is observed that is characteristic not that likely of cultures in inhibited cells, but rather, of derivative IFN groups: the largest amount of anti-proliferative activity was shown by aconite IFN derivative with 1 substituted lysine and three for SW-480 cultures. Although the succinic derivatives were less active, many of the derivatives maintained a high level of activity up through 6-7 substitutions. When a switch was made to a culture that was not sensitive to interferon, a leap was observed in maximum activity in the less acidic derivatives. It is interesting that cell division was fully inhibited just twelve hours after the introduction of acylated interferons to the culture (within the next 60 hours, the number of cells did not increase). In certain live cells, cytological changes were observed that are characteristic of apoptosis: fragmentation of the cell nucleus and karyolysis. The slower growth in more than 95% of the cells was observed after just twelve hours of incubation, whereas the control IFN in the same concentration was literally ineffective.

Some conclusions may, therefore, be drawn:
1—The acylation of one lysine in α-2b-interferon in cis-aconite and succinic anhydrides led to a 3-3.5-fold increase in the anti-proliferative activity of interferon in a sensitive culture of a CaOV ovarian cyst adenocarcinoma.
2—The largest anti-proliferative activity was demonstrated against cultures of large intestinal SW-480 cancer that have low sensitivity to native interferon by three-substituted lysine derivatives, both among the aconite and the succinic derivatives.
3—Acylated interferon leads to a broadening of the spectrum of its anti-proliferative activity in non-sensitive cultures of cancer cells.
4—The maximum anti-proliferative effect by the acylated interferons on both sensitive and insensitive cancer cell cultures arose after just twelve hours at a 95% index of anti-proliferative activity (for 1- and 3-substituted derivatives accordingly).
5—Interferons that are acylated using aconite and succinic anhydride are a new class of promising anti-proliferative substances, and they require more intense and broader study on in vivo models.

TABLE 3
Results of the Analysis of Succinimide
Derivatives of α-2b-Interferon (SIF)

| Mole Ratio IFN to | | Mr, Da | |
|---|---|---|---|
| Anhydride | Rf | Calculated | Determined |
| 1:1 | 0.490 ± 0.005 | 18359 ± 10 | 18360 ± 50 |
| 1:2 | 0.462 ± 0.005 | 18458 ± 10 | 18460 ± 50 |
| 1:3 | 0.460 ± 0.005 | 18557 ± 10 | 18560 ± 50 |
| 1:4 | 0.456 ± 0.005 | 18656 ± 10 | 18655 ± 50 |
| 1:5 | 0.442 ± 0.005 | 18755 ± 10 | 18750 ± 50 |
| 1:6 | 0.440 ± 0.005 | 18854 ± 10 | 18855 ± 50 |
| 1:7 | 0.436 ± 0.005 | 18953 ± 10 | 18920 ± 50 |
| 0 (Control interferon) | 0.492 ± 0.005 | 18260 ± 10 | 18250 ± 50 |

TABLE 4
Results of the Analysis of Aconite
Derivatives of α-2b- Interferon (AIF)

| Mole Ratio IFN to | | Mr, Da | |
|---|---|---|---|
| Anhydride | Rf | Calculated | Determined |
| 1:1 | 0.489 ± 0.005 | 18280 ± 10 | 18300 ± 50 |
| 1:2 | 0.463 ± 0.005 | 18457 ± 10 | 18470 ± 50 |
| 1:3 | 0.461 ± 0.005 | 18555 ± 10 | 18580 ± 50 |
| 1:4 | 0.455 ± 0.005 | 18634 ± 10 | 18640 ± 50 |
| 1:5 | 0.443 ± 0.005 | 18754 ± 10 | 18760 ± 50 |
| 1:6 | 0.441 ± 0.005 | 18857 ± 10 | 18860 ± 50 |
| 1:7 | 0.438 ± 0.005 | 18956 ± 10 | 18980 ± 50 |
| 0 (Control interferon) | 0.492 ± 0.005 | 18260 ± 10 | 18250 ± 50 |

### Example 2

### Design of the Antican Self-Organizing Quasi-Living System with Anticancer Properties Based on Modified Oligonucleotides

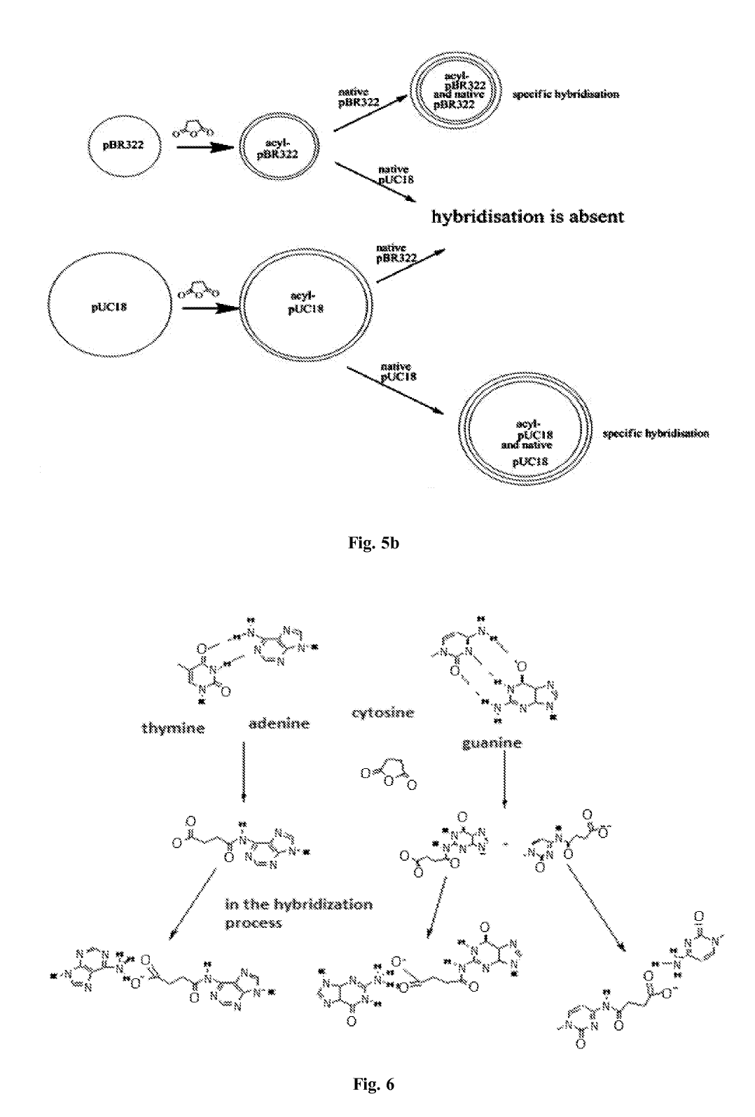

Among the existing new fields in the treatment of oncological diseases, there are several quite promising approaches. One of these approaches may be considered the development of drugs for cancer gene therapy. In this approach, the main active principle is polynucleotides. Gene therapy can be divided into two groups: means to inactivate genes and means for introducing genetic material into a cell. Gene inactivators are also called antisense polynucleotides. Many research projects conducted in this direction have not come up with truly effective in vivo drugs. This is connected with a whole host of problems: the synthesized DNA (RNA) was quickly destroyed by blood nucleases, did not penetrate the cells, and the genome repair systems were disrupted. Sometimes, a short-term block of the expression of protein targets and buildup in the hepatocytes has been observed. There are various approaches to the design of oligonucleotide antisense, but the principle of their interaction with the target remains the same: the creation of hydrogen bonds between complementary nucleotides upon increasing the level of resistance to nuclease. In certain cases, developers have protected the 5′ end of an oligonucleotide from nuclease action; in other cases, they have modified the 3′ end. AVI-biopharma's developers have exchanged deoxyribosil remnants for fragments of morpholine with the goal of creating fragments of DNA (RNA) that can withstand nuclease action. The principle of gene inactivation through their complementary interaction with antisense nucleotides has remained the same: the creation of hydrogen bonds. It is this hydrogen bond that is the main reason for the ineffectiveness of existing drugs based on antisense DNA (RNA). Ferments of a type of helicase very quickly and easily unwind antisense DNA that is hybridized with the gene target, and the gene's activity begins again. In our earlier studies, when researching acylated polynucleotides, we discovered a new phenomenon: polynucleotides that had been acylated along exocyclic amino groups hybridized selectively only with their own non-acylated precursors (FIG. 5a). The pUC18 plasmid hybridized only with its acylated derivatives, while the pBR322 plasmid hybridized, accordingly, only with acylated pBR322 (FIG. 5b). Insoluble, non-melting conjugates were created. It was the non-melting property that distinguished the classic, double-helix DNA from the hybrids that had been obtained. They would not melt at any temperature and would not dissolve in anything except in concentrated alkalis. We explain this property of the synthesized acyl-DNA by a change to the very principle of bonding between DNA chains: from hydrogen to ionic and mixed.

This bond (FIG. 6) was not stipulated at all by the natural repair mechanisms. Thus, the cell helicases and nucleases will be ineffective if hybrids are created between this type of acyl-DNA (RNA) and its non-acylated predecessors. In addition to the polynucleotide phenomenon, we also saw dependence between charge and activity in a series of other acylated biopolymers: proteins, polysaccharides, polynucleotides, tannins, bacteriophages, and immunoglobulins. On the basis of this research, a new veterinary antiviral drug was developed and implemented. We established that a certain precision modification to the structure of a biopolymer is capable of increasing its biological activity, fully changing its properties, or leading to the creation of self-organizing structures. The behaviors we discovered were taken as the basis for a principle of obtaining microRNA from acylated exocyclic amino groups. This drug was named Antican.

This microRNA is a new class of non-coded RNA with a length of 18-25 nucleotides, which negatively regulate post-translation gene expression. The mechanism of the activity of these small fragments of RNA is based on the interaction (hybridization) of microRNA with matrix RNA directly in the polyribosome complex. This hybridization will lead to the cessation of the synthesis of a specific protein. However, microRNA is not capable of penetrating a cell membrane on its own. Currently, research is being done on the creation of various transportation systems for the delivery of microRNA to cell targets.

The ability of many adenocarcinomas to pick up oligonucleotides and nanoparticles by pinocytosis from the intercellular matrix is known. Healthy cells are not capable of picking up small oligonucleotides and liposomes. This facilitates Antican aggregation selectivity in cancerous cells and the drug's lack of toxicity. In order to obtain Antican, we used accumulated RNA from yeast, fragmented pancreatic nuclease to fragments from 2 to 15 n in size. Then the exocyclic amino groups of these oligonucleotides were modified by changing their charges. For purposes of improving Antican's aggregation in tumors, it was included in monolamellar phosphatidylcholine liposomes. The selective aggregation of Antican in cancer cells leads to its hybridization with complementary targets in the matrix RNA of the cancer cell and to the gradual cessation of protein synthesis. Antican blocks not only the intronic, but also the exonic areas of matrix RNA, which leads to the practically instantaneous stoppage of protein synthesis. Antican's activity is based on the inducement of apoptosis through the stoppage of protein synthesis. Antican does not affect healthy cells, blocks the synthesis of all cell proteins, and excludes the possibility of cancerous tumors' adaptation to therapy and the selection of stable cells.

The molecular mechanism of the drug activity has not been studied.

In addition to showing anticancer activities in vitro, Antican also showed high activity in vivo on models of benzidine sarcomas in rats and Ehrlich's ascites adenocarcinoma in mice (presented later in the report).

### Example 3

### Obtaining the Substance of Antican

The microbial biomass contains up to 11% nucleic acids and may serve as a raw material in obtaining microbial RNA, on the basis of which it is possible to obtain derinates that are a mixture of oligonucleotides. The object of study at this stage was RNA taken from Saccharomyces cerevisiae yeast biomass.

Separation of Total RNA from Baker's Yeast. Four kg of compressed yeast was defrosted at room temperature. It was ground and suspended in 8 L of boiling water containing 300 g of sodium dodecal sulfate. The suspension was boiled for 40 minutes while being stirred constantly. It was then poured into steel centrifuge cups, which were quickly placed in ice and cooled to 5° C. (˜15 min.), after which they were centrifuged in a 6K15 German-made centrifuge (17000 g, 10 min, 4° C.). The sediment and a part of the gel-like interphase were removed, and the RNA that had migrated to the supernatant was separated out. This was accomplished by adding NaCl to the supernatant obtained in the previous stage, until the final concentration of 3 M was reached. After dissolving the salt, the suspension was left for 1 hour to allow the formation of sediment, which was then separated by a centrifuge at 17000 g over 10 min. The sediment was rinsed with two portions of 8 L 3 M NaCl each, suspended in 2 L of ethanol, and left overnight. The next day, the suspension was centrifuged at 17000 g over the course of 10 minutes. The sediment was dissolved in distilled water to an RNA concentration of 450 D260 U/mL (˜1.6 L). The solution was clarified by centrifuging under the same conditions, after which the RNA was precipitated from the supernatant through adding NaCl to a final concentration of 0.15 M and an equal volume of ethanol (˜2 L). The formed sediment was separated from the supernatant through centrifuging (17000 g, 10 min), cleaned in 1 L of ethanol, and dehydrated in a CaCl₂ vacuum desiccator. All operations were conducted at a temperature of 0-4° C.

### Separation of High-Polymer RNA from Baker's Yeast

### Day 1: RNA Extraction

7.5 g of sodium dodecyl sulfate were dissolved in 300 mL of water in a heat-resistant one-liter glass beaker. The solution was brought to a boil, and over a period of 5 minutes, 30 g of dry yeast were added in such a manner that the temperature of the suspension did not fall below 98° C. The suspension obtained was boiled for 40 minutes while being stirred constantly, and boiling water was added to keep the mixture at 300 mL, while the original water boiled off. At the end of the extraction, the suspension was cooled to approximately 60° C. The freshly boiled water was added to a volume of 450 mL. This was mixed, poured into a 500-mL measuring cylinder, and left to settle at a temperature of 20° C. for 22 hours.

### Day 2: RNA Desalting and Rinsing the Desalted RNA with 3 M NaCl

The cooled supernatant (280 mL) was transferred to a measured glass beaker with a volume of 500 mL. 81 g of NaCl was added to it, as was water, to make a total of 450 mL. This was mixed and left to settle at a temperature of 19° C. for 6 hours. After 6 hours, the interphase formed (250 mL) was separated, and to the desalted RNA that was divided into two fractions (one part deposit, one part supernatant) was added 3 M NaCl up to 450 mL. This was mixed and left to settle overnight at a temperature of 19° C.

### Day 3: Rinsing the Desalted RNA with 3 M NaCl

The interphase (300 mL) was separated, and 3 M NaCl was added to the desalted RNA up to 450 mL. This was mixed and left to settle at a temperature of 19° C. for 5 hours. After 5 hours, the interphase (280 mL) was separated, and 3 M NaCl was added to the desalted RNA up to 450 mL. This was mixed and left to settle at a temperature of 19° C. for 3 hours. After 3 hours, the interphase (250 mL) was separated, and 3 M NaCl was added to the desalted RNA up to 450 mL; this was mixed and left to settle overnight at a temperature of 19° C.

### Day 4: Rinsing the Desalted RNA with Alcohol

There is almost no top layer. Sediment occupies a volume of ˜100 mL. The supernatant was removed, and ethanol was added to the sediment up to a volume of 300 mL. This was mixed and left to settle at a temperature of 18° C. for five hours. After five hours, the supernatant was poured off, and a fresh portion of ethanol was added to the 140 mL of sediment up to a volume of 320 mL. This was mixed and left to settle at a temperature of 19° C. for 40 minutes, after which the supernatant was poured off. 120 mL of fresh ethanol was added to the 120 mL of sediment. This was mixed, and the supernatant was poured off after the mixture had been left to stand for 30 min. To the sediment (110 mL) was added 110 mL of fresh ethanol. This was mixed, and after 30 minutes of settling, the supernatant was poured off, and the RNA suspension was poured in a thin layer into a flat pan and air-dried at room temperature until the odor of alcohol had disappeared. The pure high-polymer RNA from the intermediate product was removed through water extraction in a cellophane dialysis bag.

### Enzymatic Splitting of the Total RNA Obtained

In the capacity of a nuclease, pancreatic ribonuclease (RNAase) with an activity of 14000 U/mg in a quantity of 0.4% of the mass of the RNA was used. The RNA splitting was conducted over a period of four hours. The hydrolyzate was dehydrated in a flash drier.

### Chemical Modification of the Hydrolyzate's Oligonucleotides

A 3.5% water solution of the hydrolyzate was obtained; succinic anhydride was added in a quantity of 10-45% of the dry weight of the hydrolyzate; this was mixed in the cold until the anhydride was fully dissolved. The solution obtained was sterilized for 120 min with flowing steam. The prepared solution was studied further for presence of anticancer properties.

In establishing the minimum concentration of Antican that could slow the growth of cells, a comparison was made between the number of surviving cells and the concentration of Antican in the solution.

TABLE 5
Effect of Antican on HeLa Cells

| | | Number of Live | | | |
|---|---|---|---|---|---|
| | | Cells after | | | |
| | | Incubation, | | Number of Live | |
| | Number of Cells | Millions, | | Cells after | |
| Dose, | before Incubation, | ±1000 | | Incubation, % | |
| μg/mL | Millions | Antican | Taxotere | Antican | Taxotere |
| 2 | 150000 ± 1000 | 72000 | 150000 | 48 | 100 |
| 4 | 153000 ± 1000 | 21400 | 150000 | 14 | 98 |
| 6 | 150000 ± 1200 | 9800 | 145000 | 6.5 | 97 |
| 8 | 152000 ± 1000 | 0 | 135000 | 0 | 89 |
| 10 | 158000 ± 1000 | 0 | 130000 | 0 | 82 |
| 12 | 162000 ± 1000 | 0 | 153000 | 0 | 94 |

As may be seen in Table 5, an effective dose of Antican is between 8-12 μg/mL solution.

Antican led to a 95% degeneration of tumor cells. To confirm the in vivo antitumor activity, Antican was studied in benzidine skin sarcoma and reinjected ascites adenocarcinomas in Barbados mice. In five mice with adenocarcinomas, the distribution of the Antican liposome throughout the animals' bodies was also studied using a fluorescent probe dissolved in a phospholipid layer.

### A Study of the Anticancer Activity of Antican on Benzidine Sarcoma

Before applying it to the silica gel, 7 mL of a solution of 2% benzidine and 0.9% sodium chloride were added until an opalescent suspension was formed (1 g silica gel for 5 mL NaCl solution). Twenty-five Barbados mice of both sexes with weight of 18-20 g that were kept on a vivarium diet were administered benzidine and phorbol acetate immobilized on silica gel subcutaneously near the neck. After two weeks, 18 animals had developed tumors of different sizes in the form of a small bump on the neck near the silica gel granulomas. Each group of animals was administered the corresponding compound parenterally, at a dose of 100 μg/kg weight, twice a day for two weeks, starting 16 days after administration of the carcinogen.

TABLE 6
Antitumor Activity of Antican in Comparison to the
Combination of an Analog (Taxotere) and Lipid

| | Weight of Animal (g) | |
|---|---|---|
| Drug Name | Before Treatment | After Treatment |
| Taxotere | 28 ± 1.2 | 23 ± 1.1 (2 mice died) |
| Antican | 25 ± 1.7 | 15 ± 1.5 |
| Bare Liposomes | 26 ± 2.1 | 34 ± 1.3 (5 mice died) |

Note:
n = 7,
p > 0.05 in comparison with the control and previous data.

As may be seen in Table 6, Antican decreased weight of the experimental animals by 10 g; weight of control animals continued to increase, and some of them died. After the dissection of the silica gel granulomas, it was established that animals treated with Antican did not show signs that the granulomas had turned into malignant sarcomas. Survival rates in animals are presented in Table 7.

TABLE 7
Survival Rates in Animals with Benzidine Skin Sarcoma

| Drug Name | Animal Survival, Days |
|---|---|
| Taxotere | 28 ± 1.1 |
| Antican | 49 ± 1.2 |
| Bare Liposomes | 17 ± 0.9 |

Note:
n = 10,
p > 0.05 in comparison with the control and previous data.

Thus, Antican prolongs life in animals twice as long as does Taxotere.

### Study of Antican Antitumor Activity in Ehrlich's Ascites Adenocarcinoma

The antitumor activity of the compositions were studied in models of Ehrlich's ascites carcinoma in young Barbados mice of both sexes with weights between 15-17 g (68 individuals), which were kept on a vivarium diet.

45 mice were inoculated from a mouse with adenocarcinoma using an insulin syringe with 0.1 mL ascitic fluid in the region of the liver. Within seven days, 42 mice showed signs of tumors (the body weight and belly size increased); two mice died on the second day; one mouse did not show signs of a tumor.

Ten mice were administered Antican (see Table 8).

TABLE 8
Qualitative Biological and Statistical Characteristics
in the Study of Antican Antitumor Activity

| | | Time of Death | |
|---|---|---|---|
| | | of Animals after the First Injection. | |
| | | Average Value | |
| | | Days | |
| Substance | Liposomes | Experimental Animals | Control Animals |
| Antican | + | 37.4 ± 0.88 | 3.2 ± 0.44 |
| -//- | − | 18 ± 3.2 | 3.1 ± 0.48 |
| Taxotere | + | 15 ± 0.5 | 3 ± 0.5 |
| -//- | − | 14 ± 0.12 | 3 ± 0.6 |

Note:
n = 10,
p > 0.05 in comparison with the control and previous data.

Antican was given to those mice from which blood was drawn. Mice with Ehrlich's adenocarcinoma, after being given the tumor and treated, lived for 18 days when administered the Antican substance, which is 6 times longer than the control, and 37 days, when administered Antican in liposomes, which is 12 times longer than the control. At an accuracy level of more than 99.5%, we can confirm significant increase in anti-carcinogenic activity in liposomal Antican over the control, Taxotere. After dissection of the animals, signs of tumors and metastasis were not found in their bodies.

### Example 4

### Method of Obtaining Self-Organizing Systems from a Base of a Group of Modified Peptides with Antiviral Properties (the Drug Albuvir)

500 mg of dry, nonfat milk is sprinkled into 50 mL of distilled water and the pH is brought to 8.0 using sodium hydroxide solution. To the obtained protein solution, trypsin immobilized on caproic granules is added at ferment: substrate ratio of 1:100 in a 0.1 M phosphate buffer solution. This is constantly stirred for four hours. Then the immobilized ferment is removed through filtration. To the supernatant fluid peptide mixture obtained, 750 mg of solid succinic anhydride is added and the substance is stirred at room temperature until the anhydride dissolves. The peptide mixture is preserved by adding benzalkonium chloride in the amount of 0.04% of the drug volume. The created mixture of peptides is lyophilized in sterile beakers and further used for the study of its antiviral activity and the confirmation of its action mechanism.

### Confirmation of Albuvir Action Mechanism

Complexes of α-β-importins are universal proteins that transport viral polynucleotides (RNA or DNA) to the cell nucleus. It has a conservative amino acid identification peptide, which recognizes nuclear pore proteins and opens them so that the nucleoprotein and importin complexes can enter. To confirm the peptide action mechanism we used DNA from the type 1 L-2 herpes viral strain. The DNA was separated using known method. The DNA was conjugated with particles of colloidal gold. The complex obtained was included in liposomes. Appropriately, the experiment was described with the SV40 virus in Old World monkeys [13].

These liposomes merged with the cell membranes from the chicken fibroblast culture. After merging of the liposome with the cell membrane, the virus's DNA entered the cytoplasm along with the gold particles. The cellular analog of the T-antigen carried the colloidal particles into the nuclear pores with the polynucleotide. If the cells were incubated with the group of peptides that are being patented, aggregation of the particles of colloidal gold in the nuclear pores was not observed. All the particles were equally distributed throughout the cells' cytoplasm. In this case, the cytopathic activity of the virus was not observed. Thus the designed drug slows the process of transportation of viral DNA to the cell nucleus, which was to be proven.

Effects of Albuvir Treatment on Mice Infected with Herpes Encephalitis

Twenty BALB-C white mice were infected intraperitoneally with a lethal dose of 0.01 mL 7.0 Ig HSV1, strain L2. After two days, the majority of the animals showed signs of encephalitis: sluggishness, reduction in food intake, and convulsions. The treatment started on the second day after infection. A group of modified peptides was administered perorally, at a dosage of 10 mg/kg animal weight, twice a day for five days. The infected animals in the control group were administered a 0.9% solution of sodium chloride. The main criterion for the effectiveness of the drug was the percentage of surviving animals in the main experimental group against the control group in 17 days. In the experimental group of animals that had been given the drug, only two out of 25 animals died (8%), while in the control group, 18 of 20 animals died (90%). In addition, on the second day of treatment with the group of modified peptides, the symptoms of herpes encephalitis had disappeared. Thus, the drug had vivid therapeutic effect in the treatment of herpes encephalitis in mice.

### Therapeutic Effect of the Drug on a Model of Ophthalmic Herpes (OH) in Rabbits

The most convenient method for creation of OH and the evaluation of the effectiveness of drugs under these conditions is the model of herpes keratitis and kerato-conjunctivitis in rabbits. The HSV-1 L2 strain was used in the form of a cultured fluid infected by a culture of Hep-2 cells. The maximum value of the infectious titer was 5.0-5.5 Ig LD50/0.03 mL. Rabbits of both sexes with a weight of 2.5 kg were used. To create OH model rabbits eyes were rinsed with saline solution and anesthetized with Ultracaine injected into the conjunctiva. HSV-1 was applied to the abraded corneas at infective dose (6-7 drops). The intensity of the clinical symptoms of the rabbits OH was evaluated using a four-point system for each symptom, which were later tallied. The evaluation of the effectiveness of the chemical drugs was conducted taking into account the difference in symptoms displayed (Intensity Level of Viral Symptomatic—LIVS in points) in the experimental and control groups (infected animals that were administered a 0.9% solution of sodium chloride and a group of animals that were administered the prototype drug). The LIVS in the control group (untreated animals) was taken to be 100%. The drug was administered 48 hours after infection, at a dose of 0.01 g drug per rabbit three times a day perorally in a solution of distilled water. Analogously, but parenterally (in an ear vein), acylated albumin from the prototype and a 0.9% NaCl solution was administered. In 90% of the rabbits, the intensity of the conjunctivitis alone rated three to four points. The duration of the symptoms was, in average, around 21 days. They achieved their maximum development in four to eight days (LIVS at a level of eight to ten points). The evaluation criteria for the therapeutic effect of the drugs were: a) a lessening in the intensity of the clinical symptoms; b) a reduction in the length of the illness; c) prevention of lethal meningoencephalitis (survival). The data on the effectiveness of the studied drugs is presented in Table 9.

TABLE 9
Treatment Effect of Albuvir in Infected Rabbits

| | Number | | LIVS |
|---|---|---|---|
| Drug Name | of Animals | Length of Illness (Days) | (Points) |
| Albuvir | 7 | 7.5 ± 2.5* | 36.3 ± 4.8 |
| Parenteral | 7 | 25.5 ± 2.5* | 100 ± 2 |
| Acylated | | | |
| Albumin | | | |
| Untreated Control | 5 | 26 ± 2.5** | 109.2 ± 2.2 |

*P < 0.001
**P < 0.01

As it is shown in the table 9, the use of Albuvir led to a 2 times decrease in the symptoms of the illness comparing with the animals that were infected but not treated. After Albuvir application, the lessening in the symptoms severity was observed, as well as reduction of the length of the illness by two thirds at a survival rate of 100%. The LIVS for the drug were less than the LIVS for the control. At the same time, the acylated albumin from the prototype administered in the same dosage parenterally (intravenously in the ear vein) did not affect the life span or the length of the illness.

### Research of the Anti-Influenza Properties of the Substance to be Patented on in Ovo Model

Strains of the Influenza-A-Hong Kong-95 H3N2 virus were used in the experiments. A total of sixty 10-11-day-old chicken embryos were used in the experiment. Twenty embryos were used for each drug: 0.1 mL of the cultured fluid with 7 Ig virus titer was introduced to the embryo amniotic cavities; they were incubated for 1 day at a temperature of 350 C. Then 0.3 mL of each experimental drug (from a calculated 0.003 g drug per kg weight) was introduced into the amniotic cavities of 20 of the infected embryos. 20 more embryos were administered 0.3 mL each of acylated albumin from the prototype, and 20 of the embryos were administered 0.3 mL each of a 0.9% NaCl solution. After three days, the embryos were dissected and a hemoagglutination reaction was initiated with a 5% suspension of erithrocytes. Allantoic and amniotic fluids were used as material in the hemoagglutination reaction. The virus titer was established through tenfold dilution in allantoic and amniotic fluids against a pure virus (Table 10).

TABLE 10
Anti-Influenza Activity of the Union
Acylated Peptides on in ovo Model

| | Virus Titer* (Ig) | | |
|---|---|---|---|
| | | | In Control (Mixture |
| | | | of Allantoic and |
| | In | In | Amniotic Fluids of |
| Drug | Allantoic Fluid | Amniotic Fluid | Non-Infected Embryos) |

| | | | |
|---|---|---|---|
| Albuvir | 1.5 ± 0.2 | 0 | 0 |
| Acylated | 7.2 ± 1.2 | 6.5 ± 1.5 | 0 |
| Albumin | | | |
| from the | | | |
| Prototype | | | |
| 0.9% Saline | 7.5 ± 1.0 | 6.5 ± 1.5 | 0 |
| Solution | | | |

*when P < 0.001

As can be seen in Table 10, the substance being patented reduces the concentration of the influenza virus by 6 Ig, while the acylated albumen does not have anti-influenza properties. In the amniotic fluid into which the drug was introduced, the virus was not found at all. It is possible that, after ending up in the cell cytoplasm, it disintegrates under the influence of proteases and nuclease. This is the evidence of another action mechanism of the acylated albumin hydrolyzate in opposition to pure acylated albumin. The latter, as an inhibitor of virus adhesion, is capable only of protecting cells from being infected by viruses, but did not block the replication of the virus in infected cells.

### REFERENCES

1. Lundstrom K. Structural genomics and drug discovery. J Cell Mol Med. 2007 March-April; 11(2):224-38.
2. Ma B, Nussinov R. Trp/Met/Phe hot spots in protein-protein interactions: potential targets in drug design. Curr Top Med Chem. 2007; 7(10):1007-13. Links
3. Keskin O, Gursoy A, Ma B, Nussinov R. Towards drugs targeting multiple proteins in a systems biology approach. Curr Top Med Chem. 2007; 7(10):945-53.
4. Shchekotikhin A. E., Dezhenkova L. G., Susova O. Y., Glazunova V. A., Luzikov Y. N., Sinkevich Y. B., Buyanov V. N., Shtil A. A., Preobrazhenskaya M. N. Naphthoindole-based analogues of tryptophan and tryptamine: synthesis and cytotoxic properties.//Bioorg. Med. Chem.—2007.—V. 15.—P. 2651-2659.
5. U.S. Pat. No. 5,495,423 General strategy for vaccine and drug design
6. U.S. Pat. No. 6,226,603 B1 Method for the prediction of binding targets and the design of ligands
7. U.S. Pat. No. 5,854,992 System and method for structure-based drug design that includes accurate prediction of binding free energy
8. U.S. Pat. No. 6,226,603 B1 Method for the prediction of binding targets and the design of ligands
9. U.S. Pat. No. 5,854,992 System and method for structure-based drug design that includes accurate prediction of binding free energy
10. Jean-Marie Lehn. Supramolecular Chemistry. Concepts and Perspectives.—Weinheim; New York; Basel; Cambridge; Tokyo: VCH Verlagsgesellschaft mbH, 1995.—P. 103 (Chapter 7)
11. Butcher L A, Tomkins J K. A comparison of silver staining methods for detecting proteins in ultrathin polyacrylamide gels on support film after isoelectric focusing. Anal Biochem 1985; 1: 384-8.
12. Melen K, Ronni T, Broni B, Krug R M, von Bonsdorff C H, Julkunen I. Interferon-induced Mx proteins form oligomers and contain a putative leucine zipper. J Biol Chem. 1992267, 25898-907.

1. A method of design and synthesis of a new drug, comprising:
determination of a biopolymer target for the drug's action;
determination of a quantity of nitrogen-containing groups that are positively charged and available for modification in the biopolymer target;
determination of a modification rate for the determined quantity of the nitrogen-containing groups;
fragmentation of the biopolymer target into oligomer fragments;
combinatorial modification of the oligomer fragments by substituting a selected number of positively charged nitrogen-containing groups for negatively charged groups, the number is selected according to the a modification rate;
application of the modified oligomer fragments as a self-assembled drug that is complementary for the biopolymer target.

2. The method according to claim 1, wherein the biopolymer target is a protein.

3. The method according to claim 1, wherein the biopolymer target is milk.

5. The method according to claim 1, wherein the biopolymer target is egg white.

6. The method according to claim 1, wherein the biopolymer target is a mixture of milk and egg white proteins.

7. The method according to claim 1, wherein the biopolymer target is DNA

8. The method according to claim 1, wherein the biopolymer target is RNA.

9. The method according to claim 1, wherein the biopolymer target is a mixture of DNA and RNA.

10. The method according to claim 1, wherein the fragmentation of the biopolymer target into oligomer fragments is effected by proteases.

11. The method according to claim 9, wherein the protease is trypsin.

12. The method according to claim 1, wherein the fragmentation of the biopolymer target into oligomer fragments is provided by nucleases.

13. The method according to claim 1, wherein the fragmentation of the biopolymer target into oligomer fragments is provided by synthetic nucleases.

14. The method according to claim 1, wherein the combinatorial modification of the oligomer fragments is provided by acylation.

15. The method according to claim 1, wherein the combinatorial modification of the oligomer fragments is provided by alkylation.

16. The method according to claim 13, wherein acylation is provided by anhydrides of polycarboxylic acid.

17. The method according to claim 14, wherein alkylation is provided by halogen-derivatives carboxylic acid.

18. The method according to claim 1, wherein the modification rate is determined by formulas:
m=(2ⁿ−1), where:
m—number of molecules or moles of the biopolymer-target, that must be modified to obtain the maximum amount of various derivatives of the biopolymer target,
n—quantity of the nitrogen-containing groups that are positively charged and available for modification in the one biopolymer target; and
k=n2⁽ⁿ⁻¹⁾, where:
k—number of moles of modifier, that is necessary for the combinatorial modification of the biopolymer target containing n groups available for modification.
